(19) United States
(12) Patent Application Publication
(10) Pub. No.: US 2023/0167397 A1
(43) Pub. Date: Jun. 1, 2023

(54) MODULAR CELL PROCESSING

(71) Applicant: CELLUNITE GMBH, München (DE)

(72) Inventor: Armin KESHMIRI, München (DE)

(73) Assignee: CELLUNITE GMBH, München (DE)

(21) Appl. No.: 17/919,829

(22) PCT Filed: Apr. 21, 2021
(86) PCT No.: PCT/EP2021/060425

§ 371 Date: Oct. 19, 2022

(30) Foreign Application Priority Data
Apr. 22, 2020 (EP) 20170943.3

(51) Int. Cl. C12M 1/36; C12M 1/00

(52) U.S. Cl. CPC C12M 41/48; C12M 45/05

(57) ABSTRACT

The present invention is directed to a system and method for modular cell processing. The invention discloses a plurality of modules that can be connected to each other generate an optimized cell processing. The connection of the two modules can comprise an exchange of volume and/or an exchange of data.

### FIELD

The present invention relates to a system and a method for modular tissue processing. The automated and/or semi-automated tissue processing can be modulated at least based on cell-relevant data.

### INTRODUCTION

The current state of the art includes various mechanical and enzymatic processes for the production of stem cell products. One is mechanical-processing: centrifuges are used for the preparation of blood and tissue products, as well for analysis or autologous therapy applications. For example, there are methods such as platelet rich plasma (PRP), fat processing, and bone marrow processing in centrifuges for the generation of a stem cell suspension (SVF). The other option is manual centrifuges.

During the centrifugation of the fat cell extract, various sources add enzymes, such as conventional collagenases, to free the fat extract from the fat cells and extracellular matrix in order to obtain a purer stem cell suspension.

Machines for automated fat tissue and bone marrow processing are also known. These are equipped for centrifugation and addition of enzymes, in a closed manner. Some of them are also configured to add enzymes by manual shaking and then performing the automated centrifugation.

For example, U.S. Pat. No. 9,144,583 B2 provides automated devices for use in supporting various cell therapies and tissue engineering methods. An automated cell separation apparatus is capable of separating cells from a tissue sample for use in cell therapies and/or tissue engineering. The cell separation apparatus can be used in combination with complementary devices such as cell collection device and/or a sodding apparatus to support various therapies. The automated apparatus includes media and tissue dissociating chemical reservoirs, filters, a cell separator and a perfusion flow loop through a graft chamber which supports a graft substrate or other endovascular device. The present invention further provides methods for using the tissue grafts and cell samples prepared by the devices described herein in a multitude of therapies including revascularization, regeneration and reconstruction of tissues and organs, as well as treatment and prevention of diseases.

Further, EP 2714887 A1 provides an automated system for isolating stromal vascular fraction cells from the mammalian tissue. The system comprises a plurality of containers for storing buffer solutions, tissue samples and digestive buffers. A tissue processing unit fluidly connected to the containers for processing the tissues. The tissue processing unit performs at least one of a washing processes, digestion process, phase separation process and combination thereof for separating an aqueous fraction of tissue and a fatty fraction. A cell concentration unit fluidly connected to the tissue processing unit for receiving the aqueous fraction of tissue from the tissue processing unit. The cell concentration unit filters the aqueous fraction of tissue by vibrating a filtration assembly by a filter vibrator. A waste collection unit fluidly connectable to the tissue processing unit and cell concentration unit is provided for receiving waste tissues. The system further comprises a control unit to control the operation of the system.

Also, U.S. Pat. No. 7,390,484 B2 is directed to cells present in processed lipoaspirate tissue that is used to treat patients. Methods of treating patients include processing adipose tissue to deliver a concentrated amount of stem cells obtained from the adipose tissue to a patient. The methods may be practiced in a closed system so that the stem cells are not exposed to an external environment prior to being administered to a patient. Compositions that are administered to a patient include a mixture of adipose tissue and stem cells so that the composition has a higher concentration of stem cells than when the adipose tissue was removed from the patient.

Additionally, CN 109082408 A discloses a device for fragmenting fat tissue, described device includes two no less than two syringes and connecting tubes, and for syringe by nipple and connecting tube connection, the connecting tube has a no less than bending structure, connecting tube minimum diameter 3 mm. After adipose tissue is crushed using device provided by the invention, the time of digestion fat stem cell and the dosage of clostridiopetidase A can significantly be reduced, only need to reach for 20 minutes good digestion effect, while the quantity of the stem cell obtained is 4 times of conventional method or more. It is repeatedly centrifuged the clostridiopetidase A for effectively eliminating digestion simultaneously, ensure that the safety for the fat stem cell being separated to, there is the value and potentiality of very big popularization and application.

Further, CN 107224614 A discloses a kind of preparation and its clinical practice of the adipose-derived stromal cells co-graft material rich in cell factor. Acquisition and purifying that preparation method is rolled into a ball by 1) lipochondrion; 2) acquisition of nanometer adipose stromal cells; 3) biological characteristics of nanometer adipose-derived stem cells (NFSCs) and multidirectional differentiation identification; 4) four steps of acquisition rich in cell factor fibrin glue prepare graft materials, reparation and correction for clinical upper surface portion, chest and other body surface soft tissue depressions deformity. The technology described promotes wound or wound repair based on stromal vascular fraction and reduces the new technology that scar proliferation, promotion organization regeneration and repair and organ are rebuild, shorten the rehabilitation duration of patient, improve quality of rehabilitation, good curative effect is obtained, the autologous adipose tissue skin grafting and mending soft tissue or organ defect, burn wound, refractory wounds and mammary cancer breast reconstruction aided in for clinically cell provides experimental basis and theories integration.

Also, KR 101489264 B1 relates to a stem cell isolation kit and a stem cell isolation method for separating stem cells from various tissues of the human body. The stem cell separation kit comprises: an upper body having an upper accommodation space and a lower accommodation space; and a second accommodation space having a conical shape at its lower end with a cross section smaller than the first accommodation space and having a cone shape; a lower main body; a separating bar capable of being raised and lowered through the upper main body and closing the upper end of the neck portion when the lower portion is lowered to block fluid communication between the upper accommodating space and the lower accommodating space; a squeezing network detachably mounted in the first receiving space of the closed lower body; and the material accommodated in the upper accommodation space may be filtered through the sieve filter to separate a substance having a predetermined size or larger. According to such a configuration, since the separated stem cells are isolated from one container to another, and a single stem cell separation kit is used to perform operations such as separation, washing and separation of stem cells, A stem cell separation kit and a stem cell separation method in which a separation operation can be performed.

KR 101893819 B1 relates to a method for quality control of stem cells using electrophoresis and cell migration assays. More particularly, the present invention relates to a method for quality control of stem cells by distinguishing stem cells from undifferentiated stem cells and bone differentiation by applying a DC electric field to a stem cell sample, and a quality control apparatus using the same. By using the method according to the present invention, it is possible to easily determine the undifferentiated stem cells and the differentiated stem cells by applying an electric field to the stem cell samples, thereby judging the quality of the stem cell samples. In particular, by applying an electric field formed by DC to an adipose-derived stem cell sample, it is possible to easily and effectively discriminate undifferentiated adipose-derived stem cells and osteocyte-differentiated stem cells.

U.S. Pat. No. 9,956,317 B2 describes methods and kits for producing cellular fractions enriched in adipose derived stem cells. Methods are provided where adipose tissue obtained from liposuction is enzymatically treated using a solution containing collagenase and divalent cations prior to the application of traditional methods of stromal-vascular fraction isolation. The enzymatic solutions may contain collagenase types I and II to a final concentration of about 0.001 mg/ml to 0.010 mg/ml. The divalent cations may be present as calcium, magnesium, and zinc chloride. The final concentration of calcium, magnesium, and zinc may range from about 0.001 to 0.1 micromolar; about 0.005 to 0.5 micromolar; and about 0.0015 to 0.15 micromolar, respectively. The enzymatic solutions may be generated using a kit where the collagenase and divalent components are held in separate containers until just prior to use. The cellular fractions isolated in this manner may be used in autologous fat grafts in therapeutic applications.

WO 2017078563 A1 relates to medical biotechnology and cell technology. Proposed is a method for isolating stromal vascular fraction of adipose tissue, which includes: decantation of a lipoaspirate; delicate washing of the lipoaspirate in buffer solutions; enzymatic treatment using a mixture of collagenases, thermolysis, dispose and trypsin at a temperature of 37° C.; centrifugation at 500-2000 g and filtration through microfilters with a pore size of 10-300 microns with the subsequent removal of fat and stromal tissue matter; repeat washing, and concentration of the cell fraction, wherein all of the stages of the process are carried out under closed system conditions. A corresponding device is configured as a sealed container consisting of two chambers, which are separated from one another by nylon microfilters with pore sizes of 10-500 microns. The device comprises fittings, provided with valves, and channels for the introduction and removal of biological tissue and a cellular end product for subsequent administration. Owing to the presence of a cocktail of mature and progenitor cells in stromal vascular fraction, which have a paracrine effect, the sterility of all of the stages of the process and the safety thereof, the claimed method can be used in medicine for the cell-based therapy and regeneration of organs and tissues, including: soft tissue and bone regeneration, and the treatment of cosmetic defects, chronic trophic and radiation ulcers, burns, Crohn's disease, multiple sclerosis, graft-versus-host reactions, myocardial infarction and strokes of different origins.

AU 2002357135 B2 is directed to a self-contained adipose-derived stem cell processing unit, comprising: a tissue collection container that is configured to receive unprocessed adipose o s tissue that is removed from a patient, wherein said tissue collection container is defined C s by a closed system; a first filter that is disposed within said tissue collection container, wherein said n first filter is configured to retain a first component of said unprocessed adipose tissue and pass a second component of said unprocessed adipose tissue, such that said first filter I separates said first component from said second component, and wherein said first C to component comprises a cell population that comprises adipose-derived stem cells and said second component comprises lipid, blood, mature adipocytes, and saline; a cell collection container, which is configured to receive said first component comprising a cell population that comprises adipose-derived stem cells from said tissue collection container, wherein said cell collection container is within said closed system; a conduit configured to allow passage of said first component comprising a cell population comprising adipose-derived stem cells from said tissue collection container to said cell collection container while maintaining a closed system; a cell concentrator disposed within said cell collection container, which is configured to facilitate the concentration of said first component comprising a cell population that comprises adipose-derived stem cells so as to obtain a concentrated population of cells that comprise adipose-derived stem cells, wherein said cell concentrator comprises a centrifuge or a spinning membrane filter; and an outlet configured to allow the aseptic removal of said concentrated population of cells that comprise adipose-derived stem cells.

DE 102013209718 B4 relates to a device for separating adult stem cells from an adipose tissue taken from a biological structure. The device has a container for receiving a substance mixture which comprises the fatty tissue and the adult stem cells. Furthermore, the device has a flushing agent supply device, a substance mixture supply device, a stem cell removal device, a flushing agent removal device and a specifically permeable membrane. In this case, the container has at least two chambers, which are separated by the at least one membrane. The substance mixture supply device and the stem cell removal device are also separated from one another by the at least one membrane.

IT GE20120034 A1 relates to the preparation and method for producing a preparation or a tissue derivative comprising mesenchymal stem cells, to be used in cellular therapy, for cosmetic treatments, for replacing a tissue or an organ, or inducing or accelerating tissue repair or regeneration. Said method provides at least the following steps: extraction of tissue containing mesenchymal stem cells, such as adipose tissue, from a cadaveric donor by liposuction process or by surgical removal of parts of adipose tissue, mechanical treatment of said tissue, said tissue, such as the adipose tissue, being composed of a fluid component comprising an oily component, a blood component and/or sterile solutions and of a solid component comprising cell fragments, cells and one or more cell macro-agglomerates of heterogeneous sizes, and said mechanical treatment step being provided for separating and removing the fluid component from the solid component, which step of mechanical treatment separating and removing the fluid component from the solid component provides an emulsion of fluid components to be generated, by mechanical stirring.

The aforementioned described processes are often cumbersome and time-consuming and result in an unsatisfying outcome. Further, the existing processes provides a low cell viability.

### SUMMARY

In light of the above, it is an object of the present invention to overcome or at least alleviate the shortcomings of the prior art. More particularly, it is an object of the present invention to provide a method and a corresponding system for modular cell processing.

The invention bears the further preferred advantages:

Due to the modularity of the cell processing system, two or more processes can be performed at the same time. A first module can include the AMFAT component which can comprise the swivel component. The swivel component can be configured for tissue washing process (“shaker”) with the quantity and/or quality measuring done by the cell quantifying component.

A second module can comprise the centrifuge component with the corresponding quantity and/or quality cell quantifying component. In both modules there can be the possibility to perform the enzymatic cleaning by the enzyme component.

Each module can also comprise one or more additive component(s). The additive component(s) can comprise an application slot for the addition of various products such as hyaluronic acid or Platelet Rich plasma. This admixture can be beneficial for the effect of cell therapy through the added growth factors from the user's own blood (PRP) and positive for the longer retention in the application area through the hyaluronic acid.

The modularity allows several process steps to be carried out simultaneously, which saves a lot of time and resources.

The modularity also enables a combination of mechanical and enzymatic methods for cell extraction in a single device.

Various processes can be used and individually combined by separate system components. This can enable the use of mechanical cell extraction and enzymatic digestion separately. The process steps can also be combined individually and performed simultaneously due to the modularity of the system.

The different process steps can be carried out in an automated process, but are separated by a controlled tissue quantity quantification (e.g. 450 ml in total, the first 300 ml for AMFAT, which is fed into the process, and the remaining 150 ml is converted into enzymatic SVF. This can offer the possibility to combine different process steps in an automated process at the same time (e.g. breast augmentation in combination with enzymatic SVF for a longer fat retention), which saves a lot of time and allows a parallel workflow. Furthermore, the cells are not reduced in the time lag between the two processes.

The terms AMFAT (autologous micro fragmented adipose tissue) or mechanic or enzymatic SVF (stromal vascular fraction) is sometimes also designated as “Millifat”, “Micrifat” and “Nanofat”.

By generating “Super SVF” with two times enzymatic processing (swiveling and centrifugation), the mechanical damage to the cells is reduced by the reduced amplitude of the shaker and centrifuge, resulting in a higher quality enzymatic SVF.

Possibility of combination with current therapies (i.e. hyaluronic acid or PRP (autologous platelet-rich plasma, enzymes))—PRP can be added directly via the additive component by means of an additional “port”—again separately at each process step. This means a variety of different processing options with additional substances.

The at least two modules can be connected by a connection port. When the at least two modules can be plugged together, the processing unit can recognize the extension and the process is automatically changed accordingly.

Below, system embodiments will be discussed. These embodiments are abbreviated by the letter “S” followed by a number. Whenever reference is herein made to “system embodiments”, these embodiments are meant.
S1. System for tissue derived cell processing comprising:
   at least one first module (1) with an AMFAT component (11) that is configured to be connected to at least one cell quantifying component (13a, 13b).
S2. System for tissue derived cell processing comprising:
   at least one second module (2) with a centrifuge component (26) that is configured to be connected to at least one cell quantifying component (23a, 23b).
S3. System according to any of the preceding embodiments comprising the first module configured to be connected to the second module (2) to generate a third module.
S4. System according to any of the preceding embodiments comprising the second module (2) that is configured to be connected to the first module (1) to generate the third module.
S5. System according to any of the preceding embodiments comprising a plurality of first modules that is configured to be connected to a plurality of the second modules.
S6. System according to any of the preceding embodiments comprising a plurality of second modules that is configured to be connected to a plurality of the first modules.
S7. System according to any one of the preceding system embodiments further comprising an inlet for feeding tissue into the AMFAT component (11).
S8. System according to any of the preceding embodiments wherein the system comprises an input pump, configured to feed tissue into the system.
S9. System according to the preceding embodiment wherein the input pump comprises at least one of: a syringe pump, and a peristaltic pump, and a roller pump.
S10. System according to any of the preceding embodiments wherein the cell quantifying component is configured to generate at least a portion of cell relevant data.
S11. System according to any of the preceding embodiments wherein the input pump is configured to generate at least a portion of cell relevant data.
S12. System according to any of the preceding embodiments wherein the system comprises a user interface configured to generate at least a portion of cell relevant data.
S13. System according to any of the preceding embodiments wherein the system is further configured to connect the first module and the second module based on cell relevant data.
S14. System according to any of the preceding embodiments wherein the system comprises a connection port configured to connect the first module and the second module.
S15. System according to any of the preceding embodiments wherein the system comprises a processing component.
S16. System according to any of the preceding embodiments wherein the processing component is connected to the connection port.
S17. System according to any of the preceding embodiments wherein the processing component is further configured to automatically recognize when the two modules are connected by the connection port.
S18. System according to any of the preceding embodiments wherein the connection port comprises a guide pin and is further configured for electric connection.
S19. System according to any of the preceding embodiments wherein the system further comprises a plurality of connections between output of the first module and input of the second module.
S20. System according to any of the preceding embodiments wherein the system further comprises a plurality of connections between output of the second module and input of the first module.
S21. System according to any of the preceding embodiments wherein the connection between the at least two modules comprise bilateral data exchange between the modules.
S22. System according to any of the preceding embodiments wherein the connection between the at least two modules comprise a continuous volume flow between the modules.
S23. System according to any of the preceding embodiments wherein the connection between the at least two modules comprise a volume exchange between the modules.
S24. System according to any of the preceding embodiments wherein the connection port is configured to connect the first module and the second module temporarily.
S25. System according to any of the preceding embodiments wherein the first module comprises an enzyme component, configured to add an enzyme in the volume flow of the system.
S26. System according to any of the preceding embodiments wherein the first module comprises a salinity component, configured to modify the salinity of the volume flow in the system.
S27. System according to any of the preceding embodiments wherein the first module comprises an additive component, configured to add at least one agent to the volume flow.
S28. System according to any of the preceding embodiments wherein the enzyme component is configured to add the enzyme to the volume flowing in a swivel component.
S29. System according to any of the preceding embodiments wherein the additive component is configured to add the agent to the volume flowing out of the swivel component.
S30. System according to any of the preceding embodiments wherein the processing component is configured to parameterize the enzyme component based on the cell relevant data.
S31. System according to any of the preceding embodiments wherein the processing component is configured to parameterize the salinity component based on the cell relevant data.
S32. System according to any of the preceding embodiments wherein the processing component is configured to parameterize the additive component based on the cell relevant data.
S33. System according to any of the preceding embodiments wherein the second module comprises a second enzyme component configured to add at least one enzyme to the volume flowing into a centrifuge component.
S34. System according to any of the preceding embodiments wherein the second module comprises a second processing component.
S35. System according to any of the preceding embodiments wherein the second module comprise a second additive component configured to add at least one agent to the volume flowing out of the centrifuge component.
S36. System according to any of the preceding embodiments wherein the cell relevant data comprises at least one of:
   i. Quantity; and
   ii. Quality; and
   iii. Vesicles; and
   iv. exosomes, cell surface proteins; and
   v. Temperature; and
   vi. Enzyme data; and
   vii. Type of cell/exosome; and
   viii. Catalyst data; and
   ix. Viscosity; and
   x. Size of the cell/exosome composition; and
   xi. Type of the cell/exosome composition.
S37. System according to any of the preceding embodiments wherein the system is further equipped with a temperature controller (3).
S38. System according to any of the preceding embodiments wherein the temperature controller (3) is configured to maintain an optimized temperature of the volume flow in the system.
S39. System according to any of the preceding embodiments wherein the optimized temperature is automatically determined by the cell quantifying component (13a 13b), preferably based on cell relevant data.
S40. System according to any of the preceding embodiments wherein the processing component is configured for bilateral data exchange with the second processing component.
S41. System according to any of the preceding embodiments wherein the system is configured to automatically switch off the at least one of processing component or second processing component after the connection.
S42. System according to any of the preceding embodiments wherein the system comprises a fourth module, the fourth module comprising the at least one of feature of the first module and/or the second module and/or the third module.
S43. System according to the preceding embodiment wherein the fourth module is configured to be connected to the first module and/or the second module and/or the third module.
S44. System according to any of the preceding embodiments wherein the cell quantifying component of the at least two connected modules are configured to transmit encrypted cell relevant data to a server.

Below, method embodiments will be discussed. These embodiments are abbreviated by the letter “M” followed by a number. Whenever reference is herein made to “method embodiments”, these embodiments are meant.
M1. Method for modular cell processing, comprising:
   i. generating cell/exosome relevant data; and
   ii. generating an activation routine of at least one module, at least based on cell relevant data.
M2. Method according to the preceding embodiment comprises connecting the at least two modules.
M3. Method according to any of the preceding embodiments comprises automatically sensing the connection of the two modules.
M4. Method according to any of the preceding embodiments comprises modifying a volume flow in the system, according to any of the preceding system embodiments, based on a connection status.
M5. Method according to any of the preceding embodiments comprises parameterizing at least one component of the at least one module based on the connection status.

Below, use embodiments will be discussed. These embodiments are abbreviated by the letter “U” followed by a number. Whenever reference is herein made to “use embodiments”, these embodiments are meant.
U1. Use of the system according to any of the preceding system embodiments for analyzing a tissue.
U2. Use of the system according to any of the preceding system embodiments for carrying out the method according to any of the preceding method embodiments.
U3. Use of the system according to any of the preceding system embodiments for orthopedics, such as arthrosis, tissue damage to cartilage.
U4. Use of the system according to any of the preceding embodiments for dermatology, such as chronic wounds, burns.
U5. Use of the system according to any of the preceding embodiments for restoration, reconstruction, or alteration, such as breast augmentation, dark circles, hair loss.
U6. Use of the system according to any of the preceding embodiments for neurology, such as Alzheimer, Parkinson's.
U7. Use of the system to extract cell composition from adipose tissue.

Below, computer related product embodiments will be discussed. These embodiments are abbreviated by the letter “C” followed by a number. Whenever reference is herein made to “computer related product embodiments”, these embodiments are meant.
C1. A computer program product comprising instructions, which, when the program is executed by a processing component, causes a processing component to perform the method steps according to any method embodiment, which have to be executed on the first module, wherein the first module is according to any system embodiment that comprises a first module that is compatible to said method embodiment.
C2. A computer program product comprising instructions, which, when the program is executed by a combination of a processing component and user interface device, cause the processing component and the user interface device to perform the method steps according to any method embodiment, which have to be executed on the first module and the user interface device, wherein the user interface device and the first module is according to any system embodiment that comprises a first module and/or the user device that is compatible to said method embodiment.
C3. A computer program product comprising instructions, which, when the program is executed by a processing component, cause the second module to perform the method steps according to any method embodiment, which have to be executed on the processing component, wherein the second module is according to any system embodiment that comprises a second module that is compatible to said method embodiment.
C4. A computer program product comprising instructions, which, when the program is executed by a cell quantifying component, cause the cell quantifying component to perform the method steps according to any method embodiment, which have to be executed on the cell quantifying component, wherein the cell quantifying component is according to any system embodiment that comprises a cell quantifying component that is compatible to said method embodiment.

Below, diagnostic kit embodiments will be discussed. These embodiments are abbreviated by the letter “D” followed by a number. Whenever reference is herein made to “diagnostic embodiments”, these embodiments are meant.
D1. A diagnostic kit comprising:
   i. at least one input component;
   ii. at least one cell quantifying component;
   iii. at least one connection port; and
   iv. at least one output component.
D2. The diagnostic kit according to the preceding embodiment comprises a disposable diagnostic kit.
D3. The diagnostic kit according to any of the preceding embodiments configured to be attached to the system according to any of the system embodiments.
D4. The diagnostic kit according to any of the preceding embodiments further comprises a saline component.
D5. The diagnostic kit according to any of the preceding embodiments comprises a first module, further comprising a swivel component, wherein the swivel component is configured to extract at least one of adipose tissue and enzyme and saline solution.
D6. The diagnostic kit according to any of the preceding embodiments comprises a second module, further comprising a centrifuge component, wherein the centrifuge component is configured to extract at least one of adipose tissue and enzyme and saline solution.
D7. The diagnostic kit according to any of the preceding embodiments wherein the connection port is configured to connect the first and the second module.
D8. The diagnostic kit according to any of the preceding embodiments comprises a additive component wherein the additive component is configured to input at least one catalyst in the output component.

The present invention will now be described with reference to the accompanying drawings, which illustrate embodiments of the invention. These embodiments should only exemplify, but not limit, the present invention.

### FIGURE DESCRIPTION

FIG. 1 schematically exemplifies a system hardware architecture in accordance with the present invention;

FIG. 2 schematically exemplifies a modular system according to an embodiment of the present invention.

FIG. 3 shows an exemplary module according to the present invention.

FIG. 4 shows an exemplary module according to the present invention.

FIG. 5 shows an exemplary module according to the present invention.

### DESCRIPTION OF PREFERRED EMBODIMENTS AS EXEMPLIFIED IN THE FIGURES

It is noted that not all the drawings carry all the reference signs. Instead, in some of the drawings, some of the reference signs have been omitted for sake of brevity and simplicity of illustration. Embodiments of the present invention will now be described with reference to the accompanying drawings.

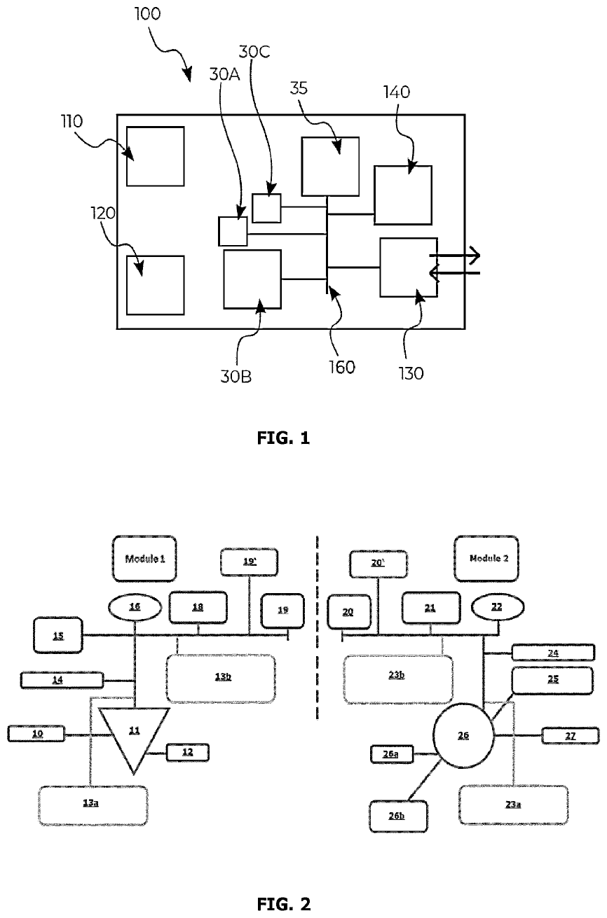

FIG. 1 provides a schematic of a computing device 100. The computing device 100 may comprise a computing unit 35, a first data storage unit 30A, a second data storage unit 30B and a third data storage unit 30C.

The computing device 100 can be a single computing device or an assembly of computing devices. The computing device 100 can be locally arranged or remotely, such as a cloud solution.

On the different data storage units 30 the different data can be stored, such as the operational parameters data on the first data storage 30A, the user data and/or cell relevant data and/or temperature data on the second data storage 30B and privacy sensitive data, such as the connection of the before-mentioned data to an individual, on the thirds data storage 30C.

Additional data storage can be also provided and/or the ones mentioned before can be combined at least in part. Another data storage (not shown) can comprise data specifying the composition or tissue and/or cell relevant data, such as volume, weight, viscocity between the different components etc. This data can also be provided on one or more of the before-mentioned data storages.

The computing unit 35 can access the first data storage unit 30A, the second data storage unit 30B and the third data storage unit 30C through the internal communication channel 160, which can comprise a bus connection 160.

The computing unit 30 may be single processor or a plurality of processors, and may be, but not limited to, a CPU (central processing unit), GPU (graphical processing unit), DSP (digital signal processor), APU (accelerator processing unit), ASIC (application-specific integrated circuit), ASIP (application-specific instruction-set processor) or FPGA (field programmable gate array). The first data storage unit 30A may be singular or plural, and may be, but not limited to, a volatile or non-volatile memory, such as a random access memory (RAM), Dynamic RAM (DRAM), Synchronous Dynamic RAM (SDRAM), static RAM (SRAM), Flash Memory, Magneto-resistive RAM (MRAM), Ferroelectric RAM (F-RAM), or Parameter RAM (P-RAM).

The second data storage unit 30B may be singular or plural, and may be, but not limited to, a volatile or non-volatile memory, such as a random access memory (RAM), Dynamic RAM (DRAM), Synchronous Dynamic RAM (SDRAM), static RAM (SRAM), Flash Memory, Magneto-resistive RAM (MRAM), Ferroelectric RAM (F-RAM), or Parameter RAM (P-RAM). The third data storage unit 30C may be singular or plural, and may be, but not limited to, a volatile or non-volatile memory, such as a random access memory (RAM), Dynamic RAM (DRAM), Synchronous Dynamic RAM (SDRAM), static RAM (SRAM), Flash Memory, Magneto-resistive RAM (MRAM), Ferroelectric RAM (F-RAM), or Parameter RAM (P-RAM).

It should be understood that generally, the first data storage unit 30A (also referred to as encryption key storage unit 30A), the second data storage unit 30B (also referred to as data share storage unit 30B), and the third data storage unit 30C (also referred to as decryption key storage unit 30C) can also be part of the same memory. That is, only one general data storage unit 30 per device may be provided, which may be configured to store the respective encryption key (such that the section of the data storage unit 30 storing the encryption key may be the encryption key storage unit 30A), the respective data element share (such that the section of the data storage unit 30 storing the data element share may be the data share storage unit 30B), and the respective decryption key (such that the section of the data storage unit 30 storing the decryption key may be the decryption key storage unit 30A).

In some embodiments, the third data storage unit 30C can be a secure memory device 30C, such as, a self-encrypted memory, hardware-based full disk encryption memory and the like which can automatically encrypt all of the stored data. The data can be decrypted from the memory component only upon successful authentication of the party requiring to access the third data storage unit 30C, wherein the party can be a user, computing device, processing unit and the like. In some embodiments, the third data storage unit 30C can only be connected to the computing unit 35 and the computing unit 35 can be configured to never output the data received from the third data storage unit 30C. This can ensure a secure storing and handling of the encryption key (i.e. private key) stored in the third data storage unit 30C.

In some embodiments, the second data storage unit 30B may not be provided but instead the computing device 100 can be configured to receive a corresponding encrypted share from the database 60. In some embodiments, the computing device 100 may comprise the second data storage unit 30B and can be configured to receive a corresponding encrypted share from the database 60.

The computing device 100 may comprise a further memory component 140 which may be singular or plural, and may be, but not limited to, a volatile or non-volatile memory, such as a random access memory (RAM), Dynamic RAM (DRAM), Synchronous Dynamic RAM (SDRAM), static RAM (SRAM), Flash Memory, Magneto-resistive RAM (MRAM), Ferroelectric RAM (F-RAM), or Parameter RAM (P-RAM). The memory component 140 may also be connected with the other components of the computing device 100 (such as the computing component 35) through the internal communication channel 160.

Further the computing device 100 may comprise an external communication component 130. The external communication component 130 can be configured to facilitate sending and/or receiving data to/from an external device (e.g. backup device 10, recovery device 20, database 60). The external communication component 130 may comprise an antenna (e.g. WIFI antenna, NFC antenna, 2G/3G/4G/5G antenna and the like), USB port/plug, LAN port/plug, contact pads offering electrical connectivity and the like. The external communication component 130 can send and/or receive data based on a communication protocol which can comprise instructions for sending and/or receiving data. Said instructions can be stored in the memory component 140 and can be executed by the computing unit 35 and/or external communication component 130. The external communication component 130 can be connected to the internal communication component 160. Thus, data received by the external communication component 130 can be provided to the memory component 140, computing unit 35, first data storage unit 30A and/or second data storage unit 30B and/or third data storage unit 30C. Similarly, data stored on the memory component 140, first data storage unit 30A and/or second data storage unit 30B and/or third data storage unit 30C and/or data generated by the commuting unit 35 can be provided to the external communication component 130 for being transmitted to an external device.

In addition, the computing device 100 may comprise an input user interface 110 which can allow the user of the computing device 100 to provide at least one input (e.g. instruction) to the computing device 100. For example, the input user interface 110 may comprise a button, keyboard, trackpad, mouse, touchscreen, joystick and the like.

Additionally, still, the computing device 100 may comprise an output user interface 120 which can allow the computing device 100 to provide indications to the user. For example, the output user interface 110 may be a LED, a display, a speaker and the like.

The output and the input user interface 100 may also be connected through the internal communication component 160 with the internal component of the device 100.

The processor may be singular or plural, and may be, but not limited to, a CPU, GPU, DSP, APU, or FPGA. The memory may be singular or plural, and may be, but not limited to, being volatile or non-volatile, such an SDRAM, DRAM, SRAM, Flash Memory, MRAM, F-RAM, or P-RAM.

The data processing device can comprise means of data processing, such as, processor units, hardware accelerators and/or microcontrollers. The data processing device 20 can comprise memory components, such as, main memory (e.g. RAM), cache memory (e.g. SRAM) and/or secondary memory (e.g. HDD, SDD). The data processing device can comprise busses configured to facilitate data exchange between components of the data processing device, such as, the communication between the memory components and the processing components. The data processing device can comprise network interface cards that can be configured to connect the data processing device to a network, such as, to the Internet. The data processing device can comprise user interfaces, such as:
output user interface, such as:
   screens or monitors configured to display visual data (e.g. displaying graphical user interfaces of the questionnaire to the user),
   speakers configured to communicate audio data (e.g. playing audio data to the user),
input user interface, such as:
   camera configured to capture visual data (e.g. capturing images and/or videos of the user),
   microphone configured to capture audio data (e.g. recording audio from the user),
   keyboard configured to allow the insertion of text and/or other keyboard commands (e.g. allowing the user to enter text data and/or other keyboard commands by having the user type on the keyboard) and/or trackpad, mouse, touchscreen, joystick—configured to facilitate the navigation through different graphical user interfaces of the questionnaire.

The data processing device can be a processing unit configured to carry out instructions of a program. The data processing device can be a system-on-chip comprising processing units, memory components and busses. The data processing device can be a personal computer, a laptop, a pocket computer, a smartphone, a tablet computer. The data processing device can be a server, either local and/or remote. The data processing device can be a processing unit or a system-on-chip that can be interfaced with a personal computer, a laptop, a pocket computer, a smartphone, a tablet computer and/or user interface (such as the upper-mentioned user interfaces).

As can be seen in FIG. 2, the system can comprise at least two modules, module 1 and module 2. The two modules can be connected via module connection 19 and module connection 20. The 2 modules can be connected by a connection port. When the two modules are plugged together, the cell quantifying component 13a, 13b, 23a, 23b can recognize the extension and the process can be automatically changed accordingly. Each of the module can comprise at least one computing unit 100.

The module 1 can comprise an AMFAT component 11. The AMFAT component 11 can include fat tissue washing process or shaking or swiveling. The amplitude for swiveling can be pre-determined using the quantified cell data by the cell quantifying component 13a, 13b, 23a, 23b. Each module can have its own cell process component 13a, 13b, 23a, 23b as shown in FIG. 2.

Further, each module can comprise its own catalyst component 18, 21. The catalyst component 18, 21 can be configured to add various products such as hyaluronic acid or Platelet Rich plasma (PRP). This admixture is beneficial for the effect of cell therapy through the added growth factors from the input sample (PRP) and positive for the longer retention in the application area through the hyaluronic acid.

Various processes from each module can be used and individually combined by separate components. For example, the AMFAT component 11 can be used without the centrifuge component 26 at first and later the centrifuge component 26 can be added to the AMFAT component 11. The process steps can also be combined individually and performed simultaneously. Again, using each individual combination, the “feedback” process is possible at any time due to the implemented quantity and quality tracker and the user-friendly software application.

The different process steps can be carried out in an automated process, but are separated by a controlled fat quantity quantification (e.g. 450 ml in total, the first 300 ml for AMFAT, which is fed into the system, and the remaining 150 ml is converted into enzymatic SVF 16. This can facilitate a possibility to combine different process steps in an automated process at the same time (e.g. breast augmentation in combination with enzymatic SVF for a longer fat retention). This can thus save a lot of time and allows a parallel workflow.

Possibility of combination with current therapies (i.e. hyaluronic acid or PRP (autologous platelet-rich plasma, enzymes))—PRP can be added directly via the catalyst component 18, 21—again separately at each process step. This means a variety of different processing options with additional substances.

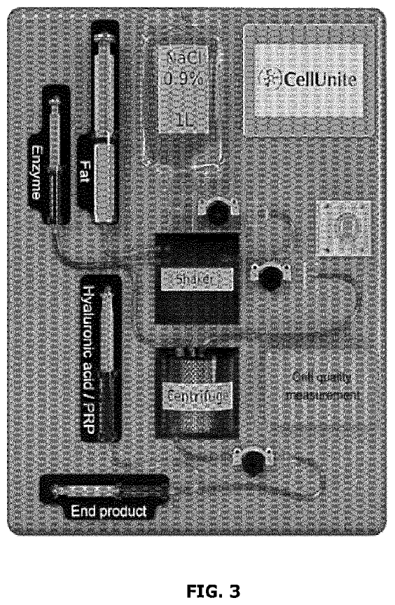

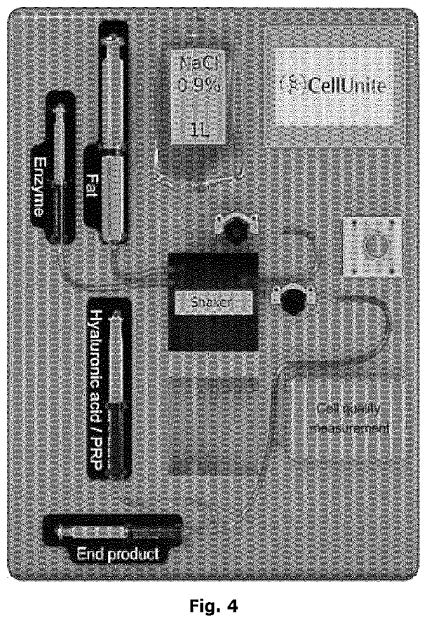

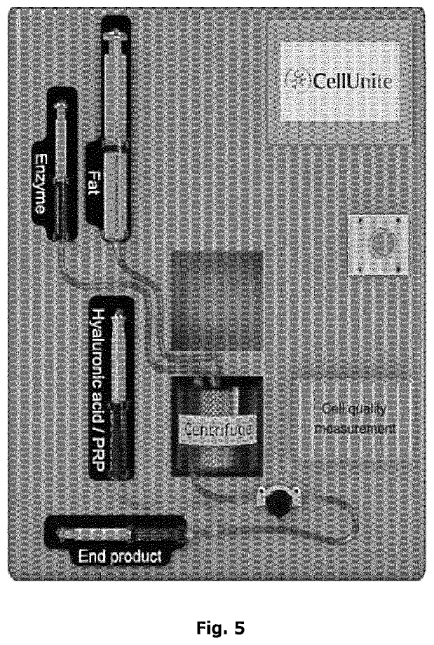

FIGS. 3, 4, 5 shows exemplary modules according to the present invention.

Reference numbers and letters appearing between parentheses in the claims, identifying features described in the embodiments and illustrated in the accompanying drawings, are provided as an aid to the reader as an exemplification of the matter claimed. The inclusion of such reference numbers and letters is not to be interpreted as placing any limitations on the scope of the claims.

The term “at least one of a first option and a second option” is intended to mean the first option or the second option or the first option and the second option.

Whenever a relative term, such as “about”, “substantially” or “approximately” is used in this specification, such a term should also be construed to also include the exact term. That is, e.g., “substantially straight” should be construed to also include “(exactly) straight”.

Whenever steps were recited in the above or also in the appended claims, it should be noted that the order in which the steps are recited in this text may be accidental. That is, unless otherwise specified or unless clear to the skilled person, the order in which steps are recited may be accidental. That is, when the present document states, e.g., that a method comprises steps (A) and (B), this does not necessarily mean that step (A) precedes step (B), but it is also possible that step (A) is performed (at least partly) simultaneously with step (B) or that step (B) precedes step (A). Furthermore, when a step (X) is said to precede another step (Z), this does not imply that there is no step between steps (X) and (Z). That is, step (X) preceding step (Z) encompasses the situation that step (X) is performed directly before step (Z), but also the situation that (X) is performed before one or more steps (Y1), . . . , followed by step (Z). Corresponding considerations apply when terms like “after” or “before” are used.

1. A system for tissue derived cell processing, comprising:
(i) at least one first module with an autologous micro-fragmented adipose tissue (AMFAT) component that is configured to be connected to at least one cell quantifying component; and
(ii) at least one second module with a centrifuge component that is configured to be connected to at least one cell quantifying component.

2. The system of claim 1 comprising the at least one first module configured to be connected to the at least one second module to generate at least one third module.

3. The system of claim 1 wherein the system comprises an input pump, configured to feed tissue into the system.

4. The system of claim 1 wherein the cell quantifying component is configured to generate at least a portion of cell relevant data, further the modules are connected based on the cell relevant data.

5. The system of claim 1 wherein the system further comprises a connection port, the connection port is configured to establish at least one of a data exchange and volume exchange between the at least one first module and the at least one second module.

6. The system of claim 1 wherein the at least one module comprises a processing component, configured to automatically recognize a connection between the at least one first module and the at least one second module.

7. The system of claim 1 wherein the system further comprises a plurality of connections between output of the at least first module and an input of the at least second module.

8. The system of claim 1 wherein the system further comprises a plurality of connections between an output of the at least second module and an input of the at least first module.

9. The system of claim 1 wherein the system is further configured to automatically turn off the at least one processing component of the at least one module after a connection.

10. The system of claim 1 wherein the cell quantifying component of the at least two connected modules are configured to transmit encrypted cell relevant data to a server.

11. The system of claim 2 wherein the system comprises a fourth module, wherein the fourth module comprises the at least one feature of the first module and/or the second module and/or the third module, further the fourth module is configured to be connected to the first module and/or the second module and/or the third module.

12. A method for modular cell processing comprising:
i. generating cell relevant data; and
ii. generating an activation routine of at least one module, at least based on cell relevant data.

13. Use of the system to carry out the method of claim 1.

14. A diagnostic kit comprising:
i. at least one input component;
ii. at least one cell quantifying component;
iii. at least one connection port; and
iv. at least one output component.

15. A computer related product for carrying out the method of claim 1.
